(19) United States
(12) Patent Application Publication
(10) Pub. No.: US 2023/0414694 A1
(43) Pub. Date: Dec. 28, 2023

(54) SALINE-BASED COMPOSITION FOR PIERCING AFTERCARE

(71) Applicant: Joonem LLC, Chatsworth, CA (US)

(72) Inventors: Eyal Termechi, Woodland Hills, CA (US); Asaf Azaria, Woodland Hills, CA (US)

(73) Assignee: Joonem LLC, Chatsworth, CA (US)

(21) Appl. No.: 17/848,290

(22) Filed: Jun. 23, 2022

(51) Int. Cl. A61K 36/886; A61K 9/08; A61K 47/02; A61K 47/36; A61K 47/22; A61K 31/14; A61K 31/197; A61K 47/46; A61K 9/00; A61M 35/00

(52) U.S. Cl. CPC A61K 36/886; A61K 9/08; A61K 47/02; A61K 47/36; A61K 47/22; A61K 31/14; A61K 31/197; A61K 47/46; A61K 9/0014; A61M 35/003

(57) ABSTRACT

A saline-based piercing aftercare solution comprising a water component, a salt component, and an anti-inflammatory component. The saline-based piercing aftercare solution can further comprise a humectant, an emollient, a preservative, an antioxidant, and one or more natural ingredients. Wherein the humectant component is sodium hyaluronate; wherein the anti-inflammatory component is aloe extract; wherein the emollient is allantoin; wherein the preservative is benzalkonium chloride; wherein the antioxidant is Vitamin B5; and wherein the one or more natural ingredients are selected from the group consisting of: rosemary, rosemary oil, rosemary extract, tea tree oil, and tee tree extract.

### FIELD OF DISCLOSURE

The present disclosure relates to topical skin-cleansing sprays applied shortly after a skin piercing. More specifically, the present disclosure relates to a composition for a saline-based piercing aftercare spray, with the intended use of cleansing, soothing, restoring, and assisting in recovering from piercings.

### BACKGROUND

Antiseptics are antimicrobial substances or compounds that are applied to living tissue to reduce the likelihood of infection. Typical examples of antiseptics are phenols and phenol derivatives, diguanides, quinolines, alcohols, peroxides, iodine and iodine derivatives, octenidine dihydrochloride, and quat salts.

While the aforementioned antiseptics may reduce rates of infection, other substances and compounds can act as milder antiseptics. One such example is common saline solution. Compared to alcohols or peroxides, saline solution is far less harsh and painful, and is less likely to dry out the area to which it is applied.

In the context of skin-piercing aftercare, skin treatment requires more than irrigation to reduce infection. Skin-piercings create, in addition to the likelihood of infection, skin inflammation and skin irritation that may be painful and reduce the quality of the skin near the piercing. For wound care, saline solution is typically not accompanied by soothing agents.

Therefore, a need exists for a topical skin-cleansing sprays applied shortly after a skin piercing with the intended use of cleansing, soothing, restoring, and helping recovery from piercings.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

One embodiment of a saline-based piercing aftercare solution may comprise a water component, a salt component, and an anti-inflammatory component. The salt component may be sodium chloride. The anti-inflammatory component may be aloe extract. The saline-based piercing aftercare solution may include a humectant. The humectant may be sodium hyaluronate. The saline-based piercing aftercare solution may include an emollient. The emollient may be allantoin. The saline-based piercing aftercare solution may include a preservative. The preservative may be benzalkonium chloride. The saline-based piercing aftercare solution may include an antioxidant. The antioxidant may be Vitamin B5. The saline-based piercing aftercare solution may have one or more natural ingredients. The natural ingredients may be selected from the group consisting of rosemary, rosemary oil, rosemary extract, tea tree oil, and tee tree extract. The water component may be in an amount from about 70 to about 99 percent by weight based on a total weight of the saline-based piercing aftercare solution. Each of the salt component, the humectant component, the anti-inflammatory component, the emollient component, the preservative component, the antioxidant component, and the one or more natural ingredients may be present in an amount from about 0.1 percent to about 10 percent by weight based on a total weight of the saline-based piercing aftercare solution.

In another embodiment, the saline-based piercing aftercare solution may comprise an emollient component, a preservative component, an antioxidant component, and one or more natural ingredients, wherein the water component is about 97.1% by weight of the total weight of the saline-based piercing aftercare solution, wherein the salt component is sodium chloride and is about 0.9% by weight of the total weight of the saline-based piercing aftercare solution, wherein the humectant component is sodium hyaluronate and is about 0.35% by weight of the total weight of the saline-based piercing aftercare solution, wherein said anti-inflammatory component is aloe extract and is about 0.2% by weight of the total weight of the saline-based piercing aftercare solution, wherein the emollient component is allantoin and is about 0.3% by weight of the total weight of the saline-based piercing aftercare solution, wherein the preservative component is benzalkonium chloride and is about 0.1% by weight of the total weight of the saline-based piercing aftercare solution, wherein the antioxidant component is vitamin B5 and is about 0.5% by weight of the total weight of the saline-based piercing aftercare solution, and wherein the one or more natural ingredients is about 0.55% by weight of the total weight of the saline-based piercing aftercare solution. The one or more natural ingredients may comprise rosemary extract and tea tree oil, wherein the rosemary extract is in an amount of about 0.45% by weight of the total weight of the saline-based piercing aftercare solution; and wherein the tea tree oil is in an amount of about 0.1% by weight of the total weight of the saline-based piercing aftercare solution.

Another embodiment, of the present disclosure is a system for delivering a saline-based piercing aftercare solution to human skin comprising a reservoir bottle, a spray nozzle, wherein the reservoir bottle comprises the saline-based piercing aftercare solution, wherein the spray nozzle may be affixed to the reservoir bottle to deliver the saline-based piercing aftercare solution to the human skin, and wherein the saline-based piercing aftercare solution comprises a water component, a salt component, a humectant component, and an anti-inflammatory component. The saline-based piercing aftercare solution may further comprise an emollient, a preservative, and an antioxidant. The saline-based piercing aftercare solution may further comprise one or more natural ingredients.

One ingredient that may improve a skin-piercing aftercare spray is aloe extract. Aloe extract is a commonly used ingredient in various acne and skin treatments. Aloe vera acts as a natural antibacterial and anti-inflammatory that reduces swelling in a given area of skin. Piercing the skin creates inflammation due to the inflow of blood to the wound site. Thus, the addition of aloe extract may soothe swelling and reduce pain.

Emollients are compounds that soften and moisturize skin, which decreases itching and flaking. An example of an emollient is allantoin. Using emollients for skin-piercing treatment may moisturize skin at the piercing site, and reduce irritation and itchiness, consequently maintaining good skin health by soothing, restoring, and helping recovery from piercings.

Various vitamins may aid in skin care and healing. One example is Vitamin B5. Vitamin B5 acts as a moisturizer which further enhances the healing process of skin wounds. Vitamin B5 further acts to soothe, calm, and repair irritated skin. Thus, Vitamin B5 and other related Vitamins may be used to soothe, restore, and help recovery from piercings.

Intense moisturizing of skin surrounding a piercing helps aid in recovery. Moisturizing compounds that deeply penetrate the skin, such as sodium hyaluronate, provide a deep hydrating effect due to the compound have a lower molecular weight than other moisturizing compounds, such as hyaluronic acid. Sodium hyaluronate is also a very stable compound, reducing the likelihood of oxidation, thus giving it a longer shelf life. Thus, compounds like sodium hyaluronate may be used to soothe, restore, and help recovery from piercings.

Since the skin surrounding piercings may be covered in topical bacteria, non-comedogenic compounds, such as rosemary oil, may be used to prevent clogged pores near a skin-piercing, to further reduce the likelihood of infection. Thus, non-comedogenic compounds, such as rosemary oil, may be used to cleanse and help in recovery from piercings.

To further aid in cleansing of a skin-piercing, other antimicrobial agents, in proper concentrations, may be useful in recovery from piercings. One example is benzalkonium chloride. When benzalkonium chloride is used in a proper concentration, it may safely act as an antimicrobial agent to prevent the growth of bacteria. Another such compound is tea tree oil. Tea tree oil when used topically is antibacterial and anti-inflammatory. Thus, these properly-balanced compounds may be used to cleanse and help in recovery from piercings.

The best way to apply the skin-piercing topical cleansing spray would be via the utilization of container with a mist-spray nozzle.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of one embodiment of a reservoir bottle topped with a spray nozzle for delivering the saline-based piercing aftercare solution to human skin.

### DETAILED DESCRIPTION OF THE PREFERRED EMBODIMENTS

In the following detailed description of various embodiments, numerous specific details are set forth in order to provide a thorough understanding of various aspects of the embodiments. However, these embodiments may be practiced without some or all of these specific details. In other instances, well-known methods, procedures, and/or components have not been described in detail so as not to unnecessarily obscure aspects of the embodiments.

While multiple embodiments are disclosed, still others will become apparent to those skilled in the art from the following detailed description. As will be realized, these embodiments are capable of modifications in various obvious aspects, all without departing from the spirit and scope of protection. Accordingly, the graphs, figures, and the detailed descriptions thereof, are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about,” may refer to a deviance of between 0.0001-40% from the indicated number or range of numbers.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are signify both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments. It is to be understood that the methods and systems are not limited to specific methods, specific components, or to particular implementations. It is also to be understood that the terminology used herein is for the purpose of describing particular embodiments only and is not intended to be limiting.

Embodiments of the present disclosure generally relate to topical skin-cleansing sprays applied shortly after a skin piercing. More specifically, the present disclosure relates to a composition for a saline-based piercing aftercare spray for all skin types, with the intended use of cleansing, soothing, restoring, and helping recovery from piercings.

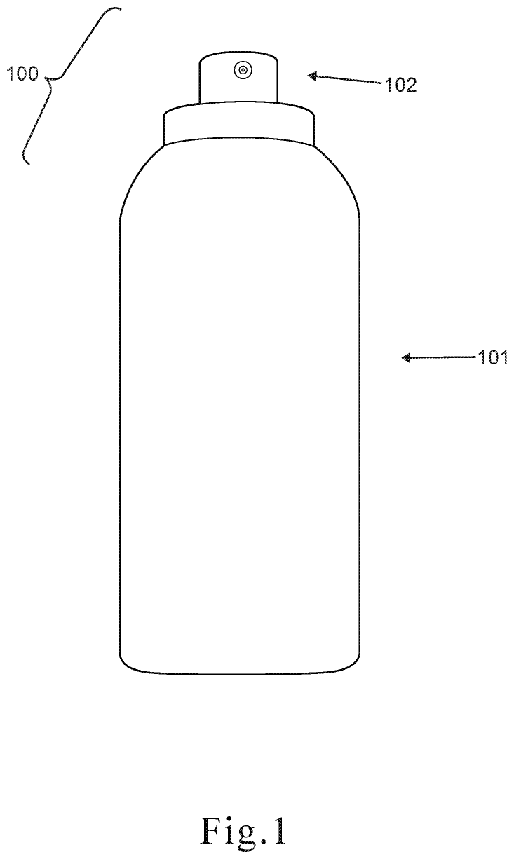

FIG. 1 is an illustration of one embodiment of a reservoir bottle 100 topped with a spray nozzle for delivering the saline-based piercing aftercare solution to human skin. In an embodiment, the reservoir bottle 101 may be a reservoir for the saline-based piercing aftercare solution. The solution may be configured to be sprayed from the spray nozzle 102.

One embodiment of the saline-based piercing aftercare solution can comprise a water component, a salt component, and an anti-inflammatory component. In some embodiments, the pH of the solution may be adjusted via the use of acidic or basic substances, such as hydrochloric acid and sodium hydroxide. In other embodiments, other acids and/or bases may be used to reach a desired pH for the solution.

In one embodiment, the water component may be about 97.1% by weight of the total weight of the saline-based piercing aftercare solution; the salt component is sodium chloride and is about by weight of the total weight of the saline-based piercing aftercare solution; the humectant component is sodium hyaluronate and is about 0.35% by weight of the total weight of the saline-based piercing aftercare solution; the anti-inflammatory component is aloe extract and is about by weight of the total weight of the saline-based piercing aftercare solution; the emollient component is allantoin and is about 0.3% by weight of the total weight of the saline-based piercing aftercare solution; the preservative component is benzalkonium chloride and is about 0.1% by weight of the total weight of the saline-based piercing aftercare solution; the antioxidant component is vitamin B5 and is about 0.5% by weight of the total weight of the saline-based piercing aftercare solution; and the one or more natural ingredients is about 0.55% by weight of the total weight of the saline-based piercing aftercare solution. The one or more natural ingredients can comprise rosemary extract and tea tree oil, wherein the rosemary extract is in an amount of about 0.45% by weight of the total weight of the saline-based piercing aftercare solution; and wherein the tea tree oil is in an amount of about 0.1% by weight of the total weight of the saline-based piercing aftercare solution. The natural ingredients can also be selected from the group consisting of rosemary, rosemary oil, rosemary extract, tea tree oil, and tee tree extract.

One embodiment of the solution may comprise one or more of water, sodium chloride, aloe extract, allantoin, Vitamin B5, sodium hyaluronate, rosemary benzalkonium chloride and tea tree oil.

Table 1 shows the weight range percentages for the components of one embodiment of the solution.

TABLE 1

| Ingredients | Percent Weight by Composition |
|---|---|
| Water | About 70% To About 99% |
| Sodium Chloride | Up To About 10% |
| Aloe Extract | Up To About 10% |
| Allantoin | Up To About 10% |
| Vitamin B5 | Up To About 10% |
| Sodium Hyaluronate | Up To About 10% |
| Rosemary | Up To About 10% |
| Benzalkonium Chloride | Up To About 10% |
| Tea Tree Oil | Up To About 10% |

Table 2 shows another set of weight ranger percentages for the components of another embodiment of the solution.

TABLE 2

| Ingredients | Percent Weight by Composition |
|---|---|
| Water | Up to 97.5% |
| Sodium Chloride | Up to 1.0% |
| Aloe Extract | Up to 1.0% |
| Allantoin | Up to 1.0% |
| Vitamin B5 | Up to 1.0% |
| Sodium Hyaluronate | Up to 1.0% |
| Rosemary | Up to 1.0% |
| Benzalkonium Chloride | Up to 0.5% |
| Tea Tree Oil | Up to 0.5% |

Table 3 shows weight percentages for the components of one embodiment of the solution.

TABLE 3

| Ingredients | Percent Weight by Composition |
|---|---|
| Water | About 97.1% |
| Sodium Chloride | About 0.9% |
| Aloe Extract | About 0.2% |
| Allantoin | About 0.3% |
| Vitamin B5 | About 0.5% |
| Sodium Hyaluronate | About 0.35% |
| Rosemary | About 0.45% |
| Benzalkonium Chloride | About 0.1% |
| Tea Tree Oil | About 0.1% |

Other embodiments of the current disclosure may contain soothing components comprising any of the following: aloe vera; calendula; chamomile and blue tansy oil; colloidal oats; evening primrose oil; niacinamide; panthenol; sea buckthorn oil; tea tree oil; rosemary; licorice; green tea; chamomile; and any combination thereof.

Other embodiments of the current disclosure may contain antioxidant components comprising any of the following: Vitamin C; Vitamin E; niacinamide; retinol; polyphenols; hyaluronic acid; resveratrol; coenzyme Q10; rosemary; tea tree oil; and any combination thereof.

Other embodiments of the current disclosure may contain surfactant components comprising any of the following: sodium lauryl sulphate; ammonium lauryl sulphate; olefin sulfonates; sodium stearate; stearic acid; cetrimonium chloride; stearalkonium chloride; disodium lauryl sulfosuccinate; cocamphocarboxyglycinate; cocoamidopropyl betaine; alpha-olefin sulfonate; and any combination thereof.

Other embodiments of the current disclosure may contain cleansing components comprising any of the following: witch hazel; cucumber and curd; honey; oatmeal; coconut oil; glycerin; hyaluronic acid; ceramides; salicylic acid; tea tree oil; and any combinations thereof.

Other embodiments of the current disclosure may contain preservative components comprising any of the following: phenoxyethanol; benzyl alcohol; sodium benzoate; potassium sorbate; ethylhexylglycerin; triclosan; methylisothiazolinone; methylchloroisothiazolinone; chlorphenesin; chloroxylenol; iodopropynyl butylcarbamate; methyldibromo glutaronitrile; and any combinations thereof.

Other embodiments of the current disclosure may contain salt components comprising sodium chloride; sea salt water; other typically used salts for these applications; and any combinations thereof.

Other embodiments of the current disclosure may contain gelling components comprising any of the following: hydroxyethyl cellulose; guar gum; xanthan gum; beeswax; gelatin; and any combinations thereof.

Other embodiments of the current disclosure may contain humectant components comprising any of the following: glycerin; urea; hyaluronic acid; salicylic acid; alpha hydroxy acids; propylene glycol; honey; sorbitol; and any combinations thereof.

Other embodiments of the current disclosure may contain emollient components comprising any of the following: lanolin; beeswax; mineral oil; patrolatum; shae butter; safflower oil; linoleic acid; oleic acid; lauric acid; stearic acid; and any combinations thereof.

Other embodiments of the current disclosure may contain anti-inflammatory components comprising any of the following: Tea Tree Oil; Rosemary; Chamomile; Aloe Vera; Witch Hazel; Calendula; CBD; Green Tea; Licorice Root; Colloidal Oatmeal; Tiger Grass; Niacinamide; Sodium Chloride; and any combinations thereof.

Other embodiments of the current disclosure may contain natural ingredients or extracts comprising any of the following: beta carotene; green tea extract; licorice; oats; soy; Vitamin C; willow herb; witch hazel; coconut oil; green tee; shea butter; tea tree oil; rosemary; aloe vera; Vitamin B5; dehydroacetic acid; and any combinations thereof.

Aloe extract may act as an anti inflammatory to reduce swelling in a given area of skin, and as a mild antibacterial to limit bacterial growth.

Emollients, such as allantoin, can be added to the solution to soften and moisturize skin, which decreases itching and flaking. Using emollients for skin-piercing treatment can moisturize skin at the piercing site, and reduce irritation and itchiness, consequently maintaining good skin health by soothing, restoring, and helping recovery from piercings.

Antioxidants, such as Vitamin B5 can be added to the solution to aid in skin repair. Vitamin B5 also acts as a moisturizer which further enhances the healing process of skin wounds.

Moisturizing compounds that deeply penetrate the skin, such as sodium hyaluronate, can be added to the solution to provide a deep hydrating effect. For sodium hyaluronate, specifically, the compound has a lower molecular weight than other moisturizing compounds, allowing for deeper skin penetration.

Since the skin surrounding piercings can be covered in topical bacteria, non-comedogenic compounds, such as rosemary oil or rosemary extract, can be used to prevent clogged pores near a skin-piercing, to further reduce the likelihood of infection.

To further aid in cleansing of a skin-piercing, other antimicrobial agents, in proper concentrations, can be useful in recovery from piercings. One example is benzalkonium chloride. When benzalkonium chloride is used in a proper concentration, it can safely act as an antimicrobial agent to prevent the growth of bacteria. Another such compound is tea tree oil. Tea tree oil when used topically is antibacterial and anti-inflammatory. Thus, these properly-balanced compounds can be used to cleanse and help in recovery from piercings.

Unless otherwise stated, all measurements, values, ratings, positions, magnitudes, sizes, locations, and other specifications, which set forth in this specification, including in the claims that follow, are approximate, not exact. They are intended to have a reasonable range, which is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. As will be realized, these embodiments are capable of modifications in various obvious aspects, all without departing from the spirit and scope of the present disclosure. Accordingly, the detailed description is to be regarded. As illustrative in nature and not restrictive. Also, although not explicitly recited, one or more additional embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A saline-based piercing aftercare solution comprising:
a water component;
a salt component; and
an anti-inflammatory component.

2. The saline-based piercing aftercare solution of claim 1, wherein said salt component is sodium chloride.

3. The saline-based piercing aftercare solution of claim 1, wherein said anti-inflammatory component is aloe extract.

4. The saline-based piercing aftercare solution of claim 1, further comprising a humectant.

5. The saline-based piercing aftercare solution of claim 4, wherein said humectant is sodium hyaluronate.

6. The saline-based piercing aftercare solution of claim 1, further comprising an emollient.

7. The saline-based piercing aftercare solution of claim 6, wherein said emollient is allantoin.

8. The saline-based piercing aftercare solution of claim 1, further comprising a preservative.

9. The saline-based piercing aftercare solution of claim 8, wherein said preservative is benzalkonium chloride.

10. The saline-based piercing aftercare solution of claim 1, further comprising an antioxidant.

11. The saline-based piercing aftercare solution of claim 10, wherein said antioxidant is Vitamin B5.

12. The saline-based piercing aftercare solution of claim 1, further comprising one or more natural ingredients.

13. The saline-based piercing aftercare solution of claim 12, wherein said one or more natural ingredients are selected from the group consisting of: rosemary, rosemary oil, rosemary extract, tea tree oil, and tee tree extract.

14. The saline-based piercing aftercare solution of claim 1, wherein said water component is in an amount from about 70 to about 99 percent by weight based on a total weight of the saline-based piercing aftercare solution.

15. The saline-based piercing aftercare solution of claim 14, wherein each of said salt component; anti-inflammatory component; humectant; emollient; preservative; antioxidant; and one or more natural ingredients is in an amount from about 0.01 percent to about 5 percent by weight based on a total weight of the saline-based piercing aftercare solution.

16. The saline-based piercing aftercare solution of claim 1, further comprising a humectant; an emollient; a preservative; an antioxidant; and one or more natural ingredients;
wherein said water component is about 97.1% by weight of the total weight of the saline-based piercing aftercare solution;
wherein said salt component is sodium chloride and is about 0.9% by weight of the total weight of the saline-based piercing aftercare solution;
wherein said anti-inflammatory component is aloe extract and is about 0.2% by weight of the total weight of the saline-based piercing aftercare solution;
wherein said humectant is sodium hyaluronate and is about 0.35% by weight of the total weight of the saline-based piercing aftercare solution;
wherein said emollient is allantoin and is about 0.3% by weight of the total weight of the saline-based piercing aftercare solution;
wherein said preservative is benzalkonium chloride and is about 0.1% by weight of the total weight of the saline-based piercing aftercare solution;
wherein said antioxidant is vitamin B5 and is about 0.5% by weight of the total weight of the saline-based piercing aftercare solution; and
wherein said one or more natural ingredients is about 0.55% by weight of the total weight of the saline-based piercing aftercare solution.

17. The saline-based piercing aftercare solution of claim 16, wherein said one or more natural ingredients comprise rosemary extract and tea tree oil;
wherein said rosemary extract is in an amount of about 0.45% by weight of the total weight of the saline-based piercing aftercare solution; and
wherein said tea tree oil is in an amount of about 0.1% by weight of the total weight of the saline-based piercing aftercare solution.

18. A system for delivering a saline-based piercing aftercare solution to human skin comprising:
a reservoir bottle;
a spray nozzle;
said reservoir bottle comprises the saline-based piercing aftercare solution;
wherein said spray nozzle can be affixed to said reservoir bottle to deliver said saline-based piercing aftercare solution to the human skin; and
wherein said saline-based piercing aftercare solution comprises a water component; a salt component; and an anti-inflammatory component.

19. The system for delivering a saline-based piercing aftercare solution to human skin of claim 18, wherein said saline-based piercing aftercare solution further comprises a humectant; an emollient; a preservative; and an antioxidant.

20. The system for delivering a saline-based piercing aftercare solution to human skin of claim 19, wherein said saline-based piercing aftercare solution further comprises one or more natural ingredients.
